(19) United States
(12) Patent Application Publication
(10) Pub. No.: US 2006/0134141 A1
(43) Pub. Date: Jun. 22, 2006

(54) Glycoconjugate vaccines containing peptidoglycan

(75) Inventors: Ali Fattom, Rockville, MD (US); Ed Hausknecht, Annapolis, MD (US); Scott Winston, Boulder, CO (US); Steve Fuller, Silver Spring, MD (US)

Correspondence Address: FOLEY AND LARDNER LLP;SUITE 500, 3000 K STREET NW, WASHINGTON, DC, 20007, US

(73) Assignee: Nabi Biopharmaceuticals

(21) Appl. No.: 11/010,563

(22) Filed: Dec. 14, 2004

(51) Int. Cl. A61K 39/085; A61K 31/739

(52) U.S. Cl. 424/243.1; 514/54

(57) ABSTRACT

The present invention relates to vaccines for treating bacterial infections, which vaccines comprise a glycoconjugate immunogen comprising at least one capsular polysaccharide conjugated to a carrier protein, such that the capsular polysaccharide contains an amount of peptidoglycan effective to improve the vaccine's properties.

### BACKGROUND OF THE INVENTION

1. Field of the Invention

The present invention relates generally to vaccines for treating bacterial infection. Specifically, the present invention provides glycoconjugate vaccines comprising a therapeutically effective amount of a capsular polysaccharide conjugated to a carrier protein, where the capsular polysaccharide comprises an amount of peptidoglycan effective to enhance properties of the vaccine, as demonstrated, for example, by improved conjugation efficiency of the capsular polysaccharide to the carrier protein or by enhanced immunogenicity of the vaccine.

2. Background of the Invention

Peptidoglycan (PG) is a heteropolymer that is unique to the bacterial cell wall and consists of a glycan backbone of alternating units of N-acetylglucosamine (GlcNAc) and N-acetylmuramic acid (MurNAc) with short peptides linked to the lactyl groups of the MurNAc moieties. In most bacterial species, the sugars are linked by β-1,4-glycosidic bonds, and the general structure of the peptide is L-alanine-D-glutamic acid-diamino acid-D-alanine-D-alanine. The di-basic amino acid in position 3 (“diamino acid” in the formula above) is typically lysine in gram positive cocci and diaminopimelic (DAP) in gram positive bacilli and gram negative bacteria. The peptide cross-linking bonds between the amino acids located on different glycan chains results in the formation of a complex three-dimensional macromolecule that forms an integral part of the bacterial cell wall. This rigid arrangement of the polymeric glycan and further cross-linked to peptides together plays a major role in determining the bacterial cell shape, maintaining the physical integrity of the bacterium. There can be subtle variations in the structure of peptidoglycans among different bacterial species, mostly relegated to the cross-linking peptides.

Numerous reports describe the pathogenic effects of peptidoglycans in animal models. Therefore, there is a desire to remove peptidoglycans from vaccines made from bacterial cell walls. For example, Simelyte et al., Infect. Immun. 68: 3535-40 (2000), disclose that a crude cell-wall preparation of gram positive bacterial cells results in chronic arthritis, when introduced into rats intraperitoneally. Similarly, Li et al., Infect. Immun. 69: 5883-91 (2001), report that intrarticular injection of a peptidoglycan preparation results in arthritic-like symptoms. Myhre et al., Infect. Immun. 72: 1311-17 (2004), characterize peptidoglycan as a major pathogenic factor in sepsis and organ injury. In a similar vein, Mattsson et al., Infect. Immun. 70: 3033-39 (2002), state that peptidoglycan is a major pathogenic factor in the induction of bacterial sepsis and endocarditis, with the concomitant activation of the procoagulant system. These reported, detrimental effects of peptidoglycan underscore conventional wisdom in the field to minimize peptidoglycan content in vaccines and other pharmaceutical preparations.

### SUMMARY OF THE INVENTION

Surprisingly, the present inventors have found that advantageous vaccine properties are associated with the presence of at least a minimum effective amount of peptidoglycan in glycoconjugate vaccines that comprise capsular polysaccharide conjugated to carrier protein. These advantages include, for example, enhanced conjugation efficiency and enhanced immunogenicity, without causing intolerable toxicity.

In accordance with one aspect of the present invention, therefore, a vaccine is provided that comprises: (A) a therapeutically effective amount of a glycoconjugate immunogen comprising at least one capsular polysaccharide and a carrier protein, wherein the capsular polysaccharide comprises a minimum effective amount of peptidoglycan, and (B) a pharmaceutically acceptable carrier for the immunogen. In one embodiment, the capsular polysaccharide comprises an amount of peptidoglycan effective to increase the conjugation efficiency of the capsular polysachharide to the carrier protein, such as, for example, by at least about 20% relative to capsular polysaccharide comprising about 2% peptidoglycan. In another embodiment, the capsular polysaccharide comprises an amount of peptidoglycan effective to increase the immunogenicity of the vaccine. In another embodiment, the capsular polysaccharide comprises at least about 5% peptidoglycan. In some embodiments, the glycoconjugate immunogen comprises one or more capsular polysaccharides expressed by Staphylococcus, such as capsular polysaccharide expressed by Staphylococcus aureus and/or capsular polysaccharides expressed by Staphylococcus epidermis. For example, the capsular polysaccharide may be a Type 5 capsular polysaccharide, a Type 8 capsular polysaccharide, a 336 capsular polysaccharide, a PS-1 capsular polysaccharide, or a combination thereof. In yet another embodiment, the carrier protein is exotoxin A from Pseudomonas, tetanus toxoid, diphtheria toxoid, alpha hemolysin, or Panton-Valentine leukocidin (PVL).

In accordance with another aspect, the present invention provides a method for treating a bacterial infection, comprising administering a vaccine that comprises (A) a therapeutically effective amount of a glycoconjugate immunogen, wherein (i) the glycoconjugate immunogen comprises at least one capsular polysaccharide and a carrier protein and (ii) the capsular polysaccharide comprises a minimum effective amount of peptidoglycan, and (B) a pharmaceutically acceptable carrier for the immunogen. In one embodiment, the capsular polysaccharide comprises an amount of peptidoglycan effective to increase the conjugation efficiency of the capsular polysachharide to the carrier protein, such as, for example, by at least about 20% relative to capsular polysaccharide comprising about 2% peptidoglycan. In another embodiment, the capsular polysaccharide comprises an amount of peptidoglycan effective to increase the immunogenicity of the vaccine. In one embodiment, the capsular polysaccharide comprises at least about 5% peptidoglycan.

In accordance with another aspect, the invention provides a method for making a vaccine comprising a glycoconjugate immunogen comprised of at least one capsular polysaccharide and a carrier protein, the method comprising (A) conjugating at least one capsular polysaccharide to a carrier protein to form a glycoconjugate immunogen, wherein the capsular polysaccharide comprises a minimum effective amount of peptidoglycan, and (B) formulating a therapeutically effective amount of the glycoconjugate immunogen with a pharmaceutically acceptable carrier for said immunogen. In one embodiment, the capsular polysaccharide comprises an amount of peptidoglycan effective to increase the conjugation efficiency of the capsular polysachharide to the carrier protein, such as, for example, by at least about 20% relative to capsular polysaccharide comprising about 2% peptidoglycan. In another embodiment, the capsular polysaccharide comprises an amount of peptidoglycan effective to increase the immunogenicity of the vaccine. In one embodiment, the capsular polysaccharide comprises at least about 5% peptidoglycan.

In accordance with yet another aspect, the present invention provides a method for enhancing the conjugation efficiency of a capsular polysaccharide to a carrier protein, comprising (i) selecting capsular polysaccharide that comprises an amount of peptidoglycan effective to contribute to enhanced conjugation efficiency of the capsular polysaccharide and carrier protein, and (ii) conjugating the capsular polysaccharide to a carrier protein. In one embodiment, the capsular polysaccharide comprises an amount of peptidoglycan effective to increase the conjugation efficiency of the capsular polysachharide to the carrier protein by at least about 20% relative to capsular polysaccharide comprising about 2% peptidoglycan. In another embodiment, the capsular polysaccharide comprises at least about 5% peptidoglycan.

In accordance with another aspect, the invention provides a method for enhancing the immunogenicity of a vaccine. The method comprises (i) selecting capsular polysaccharide comprises an amount of peptidoglycan that contributes to enhanced immunogenicity of the vaccine, (ii) conjugating the capsular polysaccharide with a carrier protein to form a glycoconjugate immunogen and (iii) preparing a vaccine comprising the glycoconjugate immunogen and a pharmaceutically acceptable carrier. In one embodiment the capsular polysaccharide comprises at least 5% peptidoglycan.

### BRIEF DESCRIPTION OF THE DRAWINGS

FIG. 1 shows the correlation between the peptidoglycan content of S. aureus Type 8 capsular polysaccharide and thiolation. Purified Type 8 capsular polysaccharides are analyzed for peptidoglycan concentration by amino acid analysis prior to conjugation to the carrier protein. The Ellman assay is used to determine the ratio of thiolation for reduced, derivatized polysaccharides.

### DETAILED DESCRIPTION OF THE PREFERRED EMBODIMENTS

As noted, the inventors have discovered that the presence of at least a minimum effective amount of peptidoglycan (PG) confers advantages to glycoconjugate vaccines comprising capsular polysaccharide (CPS) conjugated to a carrier protein. As used herein “capsular polysaccharide” includes both cell wall-associated and surface polysaccharide antigens. In one aspect, the presence of PG associated with the CPS increases the conjugation efficiency of the CPS to the carrier protein by, for example, enhancing thiolation of CPS. Enhanced conjugation efficiency offers advantages including enhancing the efficiency of the conjugation reaction (i.e., a greater percentage of the reactants become conjugated), and greater cross-linking between the CPS and the carrier protein. Greater cross-linking in turn offers advantages including larger, more immunogenic, and more stable glycoconjugate immunogen molecules. In another aspect, the presence of PG associated with CPS enhances the immunogenicity of glycoconjugate vaccines. While not wanting to be bound by any theory, the inventors believe that the conjugation of bacterial polysaccharide antigens to protein carriers alters the antigens to make them T-cell dependent immunogens, thereby potentiating the vaccine. Thus, enhancing the conjugation efficiency of the CPS to the carrier protein enhances the immunogenicity of the vaccine. Accordingly, the vaccines of the present invention are more immunogenic than prior art formulations with a lower PG content. Accordingly, the present invention provides novel vaccine formulations comprising PG and methods of making and using them.

Compositions

The present invention provides a vaccine comprising a therapeutically effective amount of a glycoconjugate immunogen comprising at least one CPS and a carrier protein, wherein the CPS comprises at least a minimum effective amount of PG, and a pharmaceutically acceptable carrier for the immunogen. While not wanting to be bound by any theory, it is believed that CPS obtained as described herein comprises PG molecules covalently bound to the CPS molecules. Alternatively, the CPS molecules may be closely associated with the PG molecules via other forces.

Capsular Polysaccharide Antigens (CPS)

As noted above, the term “capsular polysaccharide” as used herein includes both cell wall-associated and surface polysaccharide antigens. In accordance with one embodiment, the CPS is expressed by Staphylococcus, such as Staphylococcus aureus or Staphylococcus epidermidis. Exemplary S. aureus CPS include Type 5 capsular polysaccharides, Type 8 capsular polysaccharides, and Type 336 capsular polysaccharides. Exemplary S. epidermidis antigens include PS-1 capsular polysaccharides. A vaccine of the invention may comprise CPS of one or more of these types. Other CPS, such as other bacterial capsular polysaccharide cell wall antigens also can be used in accordance with the invention, alone or in combination with other antigens, such as the Staphylococcal antigens described above.

Surveys have shown that approximately 85-90% of S. aureus isolates are CPS Type 5 or Type 8. Normal individuals vaccinated with a vaccine containing both Type 5 and Type 8 capsular polysaccharide antigens are protected from infection by 85-90% of S. aureus strains. Thus, in accordance with one embodiment of the invention, the vaccine comprises glycoconjugates of both Type 5 and Type 8 CPS.

While the chemical composition of S. aureus Type 5 and Type 8 CPS is identical, the structures are different. Both are polymers composed of N-acetyl-mannuronic acid (ManNAcAPp) and N-acetyl-fucosamine (FucNAcp) in a 1:2 ratio, but they differ in the glycosidic linkages between these sugars and the site and degree of O-acetylation. Moreau et al., Carbohydr. Res., 201(2):285-97 (1990); Fournier et al., Ann. Inst. Pasteur Microbiol., 138(5):561-7 (1987). Both have FucNAcp in their repeat unit as well as ManNAcAp, which can be used to introduce a sulfhydryl group. The structures of Types 5 and 8 polysaccharide antigens have been elucidated by Moreau et al., Carbohydr. Res. 201:285 (1990); and Fournier et al., Infect. Imm. 45:87 (1984), and are shown below:

Type 5:

→4)-β-D-ManpNAcA(30Ac)-(1→4)-α-L-FucpNAc-(1→3)-β-D-FucpNAc-(1→

Type 8:

→3)-β-D-ManpNAcA(40Ac)-(1→3)-α-L-FucpNAc-(1→3)-β-D-FucpNAc-(1→

Despite the structural similarities, no demonstrable immunological cross-reactivity between the two types has been found.

Another Staphylococcus antigen that can be used in a vaccine according to the invention is the 336 CPS described in U.S. Pat. No. 5,770,208 and No. 6,194,161. This negatively-charged antigen comprises GlcNAc and 1,5-poly (ribitol phosphate) components and contains no O-acetyl groups. An exemplary 336 antigen specifically binds with antibodies to S. aureus Type 336, deposited under ATCC 55804. Staphylococcus aureus strains that carry this antigen account for nearly all of the clinically significant strains of S. aureus that are not Type 5 or Type 8 strains. Thus, the present invention contemplates, inter alia, a vaccine that comprises glycoconjugates of Type 5, Type 8, and 366 CPS, respectively. Such a vaccine may protect against infection by nearly 100% of S. aureus strains.

There are many clinically significant strains of S. epidermidis. A vaccine for the treatment or prevention of infection by S. epidermidis strains may comprise a conjugate comprising the Type 1 antigen disclosed in U.S. Pat. Nos. 5,961,975 and 5,866,140. This antigen, also referred to a PS-1, is an acidic polysaccharide antigen that can be obtained by a process that comprises growing cells of an isolate of S. epidermidis that agglutinates antisera to ATCC 55254 (a Type I isolate), extracting polysaccharide antigen from the cells to produce a crude extract of polysaccharide antigen, purifying this crude extract to produce purified antigen that contains at least a minimum effective amount of peptidoglycan, as described in more detail below; loading the purified antigen on a separatory column and eluting it with a NaCl gradient; and identifying fractions containing the polysaccharide antigen using antibodies specific to a Type I isolate.

Yet another Staphylococcus antigen useful in a vaccine according to the present invention is described in WO 00/56357. This antigen comprises amino acids and a N-acetylated hexosamine in an α configuration, contains no O-acetyl groups, and contains no hexose. It specifically binds with antibodies to a Staphylococcus strain deposited under ATCC 202176. Amino acid analysis of the antigen shows the presence of serine, alanine, aspartic acid/asparagine, valine, and threonine in molar ratios of approximately 39:25:16:10:7. Amino acids constitute about 32% by weight of the antigen molecule

Carrier Proteins

Bacterial capsular polysaccharide antigens are generally poor immunogens. Thus, they often are conjugated to a carrier protein to enhance their immunogenicity. Suitable carrier proteins according to the present invention include tetanus toxoid and diphtheria toxoid and recombinantly produced, genetically detoxified variants thereof, Staphylococcal endotoxin or toxoid, Pseudomonas aeruginosa Exotoxin A or its derivatives, including recombinantly-produced non-toxic mutant strains of Pseudomonas aeruginosa Exotoxin A, as described, for example, in Fattom et al., Inf. and Imm. 61: 1023-32 (1993), as well as other proteins, peptides and virus-like particles suitable for use as immunocarriers. Other suitable carrier proteins for use in the present invention include Staphylococcus aureus exotoxins, such as alpha hemolysin (alphatoxin) and Panton-Valentine leukocidin.

Exotoxin A is a major virulence factor from Pseudomonas aeroginosa, see Callahan et al., Infect. Immun. 43: 1019-26 (1984), and may generate toxin-neutralizing antibodies as a by-product of the vaccine of the present invention. As such, vaccines described herein, comprising Staphylococcal CPS conjugated to Exotoxin A, may be useful for patients who are at risk for Pseudomonas as well as Staphylococcus infections. See also Pollack et al., J. Clin. Invest. 63: 276-86 (1979), and Cryz et al., Rev. Infect. Dis. 9 (Suppl 5): S644-S649 (1987). A recombinant, non-toxic version of this protein (rEPA) was obtained by deleting the glutamic acid at position 553 in the enzyme active site. Lukac et al., Infect. Immun. 56: 3095-98 (1988). This deletion rendered the whole protein devoid of enzymatic activity while still maintaining the antigenicity of the native toxin. Accordingly, a vaccine within the present invention may comprise glycoconjugates comprising rEPA as a carrier protein.

Minimum Effective Amount of Peptidoglycan

Pursuant to the present invention, a vaccine can contain a glycoconjugate immunogen comprised of CPS, conjugated to a carrier protein, that comprises at least a minimum effective amount of PG. The “minimum effective amount” of PG is an amount effective to improve the properties of the vaccine. In one embodiment, an improved property is reflected by enhanced conjugation efficiency, and the “minimum effective amount” denotes a quantity of PG that is sufficient to enhance conjugation of the CPS to a carrier protein. In another embodiment, an improved property is reflected by increased immunogenicity, and the phrase “minimum effective amount” denotes a quantity of PG that is sufficient to increase the immunogenicity of the vaccine.

For example, the glycoconjugate immunogen may comprise CPS that comprises an amount of PG effective to increase the conjugation efficiency of the CPS to the carrier protein by, for example, at least about 20% relative to CPS comprising about 2% PG (i.e., a conjugation efficiency of 1.20 times the comparative CPS). Alternatively, the glycoconjugate immunogen may comprise CPS that comprises an amount of PG effective to increase the immunogenicity of the vaccine. Of course, the glycoconjugate immunogen may comprise CPS that comprises an amount of PG effective to increase both the conjugation efficiency and the immunogenicity of the vaccine.

As used herein, the phrase “conjugation efficiency” relates to the conjugation of the CPS to the carrier protein. Enhanced conjugation efficiency may be reflected in a greater percent of CPS that becomes conjugated to carrier protein during the conjugation process. For example, in accordance with the present invention at least 50% of CPS molecules are conjugated to carrier protein by the conjugation processes described below. Alternatively, enhanced conjugation efficiency may be reflected in increased cross-linking between the CPS and the carrier protein. Increased cross-linking generally results in larger glycoconjugate immunogen molecules, which generally exhibit greater immunogenicity. Additionally, increased cross-linking generally results in more stable glycoconjugate immunogen molecules.

The conjugation efficiency of a given CPS preparation may be determined by methods known in the art, including the methods described below and illustrated in the examples. As used herein, conjugation efficiency can be determined by measuring thiolation efficiency of the CPS, as illustrated below and in FIG. 1. Thus, the definition of “minimum effective amount” of PG provided herein includes a quantity of PG that is sufficient to enhance thiolation of the CPS. For example, the glycoconjugate immunogen may comprise CPS that comprises an amount of PG effective to increase the thiolation efficiency of the CPS by at least about 20% relative to CPS comprising about 2% PG (i.e., a thiolation efficiency of 1.20 times the comparative CPS).

The immunogenicity of a given vaccine preparation may be determined by methods known in the art, including the methods described below and illustrated in the examples.

The PG content of CPS can be expressed in terms of w/w % of certain amino acids, determined via amino acid analysis (AAA), conducted as follows:

A 1 mg/mL sample of purified CPS in water is hydrolyzed with vapor phase hydrochloric acid. Reconstituted primary and secondary amino acids are converted to stable fluorescent derivatives that fluoresce strongly at 395 nm. Analysis of the re-suspended protein hydrolysate is performed by reverse phase HPLC. The amino acids are quantified by means of external and internal standards. Amino acids present in the polysaccharide solution arise from (1) PG (residues Ala, Glx, Gly and Lys) and (2) residual proteins (residues Arg, Asx, Ile, Leu, Met, Phe, Ser, Thr, Thy, Val, His and Pro). Two amino acids (Cys and Trp) are not quantitated and are therefore not reported. The concentrations of the amino acids associated with PG and residual protein are reported as a mass percentage relative to the CPS using the following equations:
(Gln/Glu)+(Gly)+(Ala)+(Lys)=(PG)
[PG]×100=% Peptidoglycan
[cPS]
Σ(Amino acids)=(peptides)_{cps}
(peptides)_{cps}−(PG)/(cPS)×100=% RP
where:
[protein]=protein concentration of sample (mg/mL) by AAA
[PG]=calculated peptidoglycan content of sample (mg/mL)
[CPS]=known polysaccharide concentration of sample (mg/mL)
(peptides)_{cps}=Total peptide concentration
% RP=Residual protein (%)

In one embodiment, the CPS comprises at least about 5% PG, such as at least 5% PG, including at least about 7% PG, at least about 9% PG, and at least about 11% PG. Other amounts of PG that are effective are at least about 13% PG, at least about 15% PG, at least about 17% PG, at least about 19% PG, at least about 21% PG, at least about 23% PG, at least about 25% PG, and at least about 27% PG. The phrase “at least about” includes a percentage of PG within 1% above or below the specified amount. Thus, “at least about 5%” embraces 4-6% PG. The phrase “at least” includes a percentage of PG greater than or equal to the specified amount. Thus, “at least 5%” connotes 5% or more PG.

For example, contemplated in the present invention is a vaccine comprising a glycoconjugate immunogen that comprises at least one capsular polysaccharide and a carrier protein, wherein the capsular polysaccharide comprises at least about 5% (w/w) peptidoglycan, based on the weight of the capsular polysaccharide, and wherein the carrier protein is an alpha hemolysin, Panton-Valentine leukocidin, exotoxin A from Pseudomonas, tetanus toxoid or diphtheria toxoid, and a pharmaceutically acceptable carrier.

In another embodiment of the invention, the vaccine comprises glycoconjugates of two or more clinically significant CPS types, such as S. aureus Type 5, Type 8 and/or 366, conjugated to a non-toxic carrier protein, such as recombinant exoprotein A (rEPA). In one such embodiment, at least one of the CPS antigens comprises at least a minimum effective amount of PG, such as at least 5% PG determined as described above. In another such embodiment, each CPS antigen comprises at least at least a minimum effective amount of PG, such as 5% PG. In yet another embodiment, the glycoconjugates as a whole comprise a minimum effective amount of PG, such as a PG content of at least 5% overall, based on the total weight of all CPS antigens.

Selecting a minimum effective amount of PG may require balancing toxicity attributed to PG against the improved efficacy of the vaccine (i.e., enhanced conjugation efficiency and immunogenicity) that the PG provides. A requirement that is endemic to the field of vaccines is the mandate to balance toxicity against efficacy, and those skilled in the field can strike this balance, in a given circumstance, by way of routine experimentation. Toxicity can be determined via well-known techniques, for instance, with a focus on the reported pathogenic effects of PG, discussed above. Efficacy likewise can be determined in accordance with known methodology, as illustrated in the examples below. Thus, efficacy can be measured in terms of immunogenicity, i.e., the ability to induce antibody production, or in terms of enhanced conjugation efficiency, i.e., the ability to enhance the conjugation of CPS to carrier protein. An effective amount of PG provides a glycoconjugate vaccine that a clinician would deemed to be toxicologically tolerable but still efficacious. For instance, a clinician may find that a vaccine according to the invention, comprising CPS with a PG content ranging from at least about 5% up to and including at least about 27%, such as at least about 19%, offers enhanced efficacy (i.e., enhanced conjugation efficiency and/or immunogenicity) without exhibiting unacceptable toxic effects.

Methods

The present invention also provides methods for making the inventive glycoconjugate vaccines and methods of using them. Methods for enhancing conjugation efficiency of a CPS to a carrier protein, methods for enhancing the immunogenicity of a glycoconjugate vaccine, and methods for treating or preventing bacterial infection are specifically described.

Methods for Making a CPS Conjugate Vaccine and Related Methods

In one aspect, the present invention provides a method for making a glycoconjugate vaccine comprising CPS with at least a minimum effective amount of PG. The method comprises conjugating at least one CPS antigen to a carrier protein to form a glycoconjugate immunogen, where the CPS antigen comprises at least a minimum effective amount of PG. A therapeutically effective amount of the glycoconjugate immunogen is formulated with a pharmaceutically acceptable carrier for the immunogen to yield the vaccine. In one embodiment, the amount of PG is effective to enhance conjugation efficiency by, for example, at least about 20% relative to CPS comprising 2% PG. In another embodiment, the amount of PG is effective to increase the immunogenicity of the vaccine. In another embodiment, the CPS comprises at least about 5% PG.

For example, described herein is a method for making a vaccine comprising a glycoconjugate immunogen comprising at least one CPS and a carrier protein, wherein the capsular polysaccharide comprises a minimum effective amount of PG, and wherein the carrier protein is an alpha hemolysin, Panton-Valentine leukocidin, exotoxin A from Pseudomonas, tetanus toxoid or diphtheria toxoid, and a pharmaceutically acceptable carrier. In one embodiment, the minimum effective amount of PG is at least about 5% PG based on the weight of the CPS.

In another aspect, the invention provides a method for enhancing the immunogenicity of a vaccine. The method comprises selecting CPS with at least a minimum effective amount of PG to contribute to enhanced immunogenicity of the vaccine, and conjugating the CPS to a carrier protein to form a glycoconjugate immunogen. A therapeutically effective amount of the glycoconjugate immunogen is formulated with a pharmaceutically acceptable carrier for the immunogen to yield the vaccine. In one embodiment, the CPS comprises at least about 5% PG.

In yet another aspect, the invention provides a method for enhancing the conjugation efficiency of a CPS to a carrier protein. The method comprises selecting CPS with at least a minimum effective amount of PG to contribute to enhanced conjugation efficiency and conjugating the CPS to a carrier protein. In one embodiment, the amount of PG is effective to enhance conjugation efficiency by, for example, at least about 20% relative to CPS comprising 2% PG. In another embodiment, the CPS comprises at least about 5% PG.

Purified CPS (comprising PG) suitable for use in these methods may be obtained, for example, by treating a bacterium to release CPS, and then purifying the CPS. This process may comprise enzyme digestion of the bacterium (using, for example, lysostaphin, RNAse and/or DNAse) and recovery of CPS (using, for example, ethanol precipitation, centrifugation and filtration). Further purification steps may include dialysis (for example, to remove traces of ethanol), secondary enzyme digestion (using, for example, RNAse, DNAse and/or a protease, such as Pronase E) and dialysis, further recovery of CPS (using for example, ethanol precipitation, centrifugation, dialysis and filtration), and chromatographic methods such as ion exchange chromatography and/or size exclusion/gel filtration chromatography.

The PG content of the resulting purified CPS can be controlled, for example, at the enzyme digestion step(s). For example, adjusting the amount of lysostaphin used to release the CPS from the bacterium can affect the PG content of the CPS, with a lower lysostaphin concentration generally resulting in a higher PG content. In one embodiment, the lysostaphin concentration used ranges from about 100 μg to about 1000 μg of lysostaphin per gram of cell paste (approximately equivalent to about 7 to about 64 units/gram of cell paste). In another embodiment about 225 μg of lysostaphin per gram cell paste (approximately about 16 units/gram of cell paste) is used. Similar amounts of lysostaphin may be used in the optional second lysostaphin step.

In one embodiment, bacterial cultures are fermented and centrifuged to obtain a cell paste. Lysostaphin is added at a concentration of, for example, about 16 units lysostaphin per gram paste, to achieve the first enzyme digestion step of the process described above. In another embodiment, lysostaphin is added a second time during the purification process. For example, about 16 units of lysostaphin per gram paste may be added after the first recovery step (ethanol precipitation).

These amounts of lysostaphin are illustrative only and can be adjusted, in accordance with the target PG content. As described above, increasing the lysostaphin concentration generally will result in a lower PG content and reducing the lysostaphin concentration generally will result in a greater PG content. Also the PG content can be control by adjusting the temperature of the lysostaphin enzymatic digestion, with 37° C. being the optimal temperature and lower temperatures in the range of 20-30° C. leading to a prolonged enzymatic digestion. Thus, conducting the lysostaphin enzymatic digestion at a lower temperature can yield a formulation with a higher PG content.

The PG content of the resulting, purified CPS also can be controlled by adjusting the amount of protease used in the (optional) second enzyme digestion step, or by omitting the use of protease in that step.

For example, CPS generally can be isolated and purified from the bacterium according to the methods described in U.S. Pat. No. 6,194,161, Fattom et al., Vaccine 13: 1288-93 (1995), and Fattom et al., Infect. Immun. 58: 2367-74 (1990). In order to achieve CPS with at least a minimum effective amount of PG, however, a lower concentration of lysostaphin is used during the enzyme treatment step than is described in these publications, such as about 7 to about 64 units/gram cell paste. Additionally, the pronase (protease) step disclosed in these publications may be modified or omitted, to achieve CPS with at least a minimum effective amount of peptidoglycan, pursuant to the present invention.

Purified CPS, such as those from Staphylococcus aureus Type 5 and 8 serotypes, can be analyzed using two dimensional NMR to asses PG content. For example, analysis of the NMR spectra may denote the presence of alanine, glutamine/glutamic acid and lysine, the main amino acid components of PG. Likewise, the NMR spectra of de-O-acetylated Type 5 and Type 8 CPS may provide evidence of two unsubstituted hydromethyl groups, which would be consistent with the presence of the β-GlcNAc and β-MurNAc residues in PG.

CPS with at least a minimum effective amount of PG is selected for use in the methods of the invention. The PG content of the purified CPS can be determined using AAA as set forth above.

After isolation and purification of the CPS, CPS comprising at least a minimum effective amount of PG is conjugated to a carrier protein. In one embodiment, the carrier protein is derivatized by methods known in the art to facilitate conjugation. The CPS antigen also may be derivatized by methods known in the art to facilitate the conjugation. For example, activated carboxylate groups on the CPS antigen can be derivatized with ADH, cystamine or PDPH, and then the CPS antigen can be conjugated to the carrier protein either by a carbodiimide-mediated reaction of the partially amidated antigen to a carboxylate group on the carrier protein or by disulfide interchange of thiolated CPS antigen with an SPDP-derivatized carrier protein. Hydroxyl groups on the antigen can be activated using cyanogen bromide or 1-cyano-4-dimethylamino-pyridinium tetrafluoroborate, and then the antigen can be derivatized with the six carbon bifunctional spacer adipic acid dihydrazide (ADH), according to techniques known in the art, according to the method of Kohn et al., FEBS Lett. 154: 209:210 (1993).

In one embodiment, derivatization of the CPS is achieved by a process comprising thiolation. Such a process can be effected through the amidation of the CPS with a diaminodisulfide (cystamine). This reaction is called “thiolation” because it introduces a disulfide bond. Amidation of the carrier protein may be carried out in parallel with an activated carboxyl-terminated linker, N-succinimidyl 3-(2-pyridyldithio)propionate (SPDP). Conjugation is achieved when free thiols generated through the reduction of the thiolated CPS with dithiothreitol (DTT) are added to the SPDP-derivatized carrier protein to displace the pyridine thione and form a disulfide bond between the CPS and the carrier protein through the linker. Typically, the resulting glycoconjugate is recovered by filtration.

The ratios of SPDP derivatization of the carrier protein and thiolation of the CPS may be optimized so as to preserve the antigenic determinants on the carrier protein and the CPS, and to produce a more stable and/or more immunogenic conjugates. The amount of free thiols also may be controlled and optimized so that the chosen substitution density minimizes cross-linking of the thiolated CPS and favours the CPS-protein conjugation.

For example, an initial derivatization reaction can be conducted with a 1:1 ratio of each reactant. The antigenicity of the resulting product can be assessed by immunizing animals with the product and determining the immunogenic response by routine methods. If desired, additional derivatization reactions can be conducted with different ratios of reactants to optimize the properties of the resulting product.

As discussed above, the derivatized CPS antigen can be linked to any suitable carrier protein, such as diphtheria toxoid (DTd), recombinant exoprotein A from Pseudomonas aeruginosa (rEPA), tetanus toxoid (TTd), alpha hemolysin, Panton-Valentine leukocidin (PVL) or another suitable carrier protein by 1-ethyl-3-(3-dimethylaminopropyl) carbodiimide (EDAC). The resulting conjugates can be separated from non-conjugated CPS antigen by size exclusion chromatography.

Regardless of the method used to conjugate the CPS antigen to the carrier protein, covalent linking of the CPS antigen to the carrier protein significantly enhances the immunogenicity of the CPS antigen. This has been observed, for example, as increased levels of antibody induced to the antigen after both first and second vaccine boosts in mice. Using CPS with at least a minimum effective amount of PG in accordance with the invention therefore enhances the conjugation efficiency of the CPS to the carrier protein and enhances the immunogenicity of the vaccine.

An Ellman assay may be used to assess conjugation efficiency as measured by thiolation efficiency, i.e., to determine the ratio of thiolation of the reduced, derivatized CPS by measuring the remaining free sulfhydryl groups. For example, free sulfhydryl groups can be quantified by reacting the reduced, derivatized CPS sample or control with 5,5′-dithiobis (2-nitrobenzoic acid) (DTNB) for five minutes at room temperature. This reaction produces a mixed disulfide and 2-nitro-5-thiobenzoic acid (TNB). The concentration of TNB may be quantified by absorbance at 412 nm, with a molar extinction coefficient of 13,600. The result can be reported as the molar ratio of free sulfhydryl groups per polysaccharide (PS) trisaccharide repeat unit.

As shown in FIG. 1, CPS comprising about 5% PG has a thiolation efficiency of at least about 1.20 times that of CPS comprising about 2% PG. Thus, the CPS comprising about 5% PG exhibits an increased conjugation efficiency of 25% relative to that of CPS comprising 2% PG.

A vaccine according to the invention typically comprises a pharmaceutically acceptable carrier for the glycoconjugate immunogen. A pharmaceutically acceptable carrier is a material that can be used as a vehicle for the glycoconjugate because the material is inert or otherwise medically acceptable, as well as compatible with the active agent, in the context of vaccine administration. In addition to a suitable excipient, a pharmaceutically acceptable carrier can contain conventional vaccine additives like diluents, adjuvants and other immunostimulants, antioxidants, preservatives and solubilizing agents. For example, polysorbate 80 may be added to minimize aggregation and act as a stabilizing agent, and a buffer may be added for pH control. The vaccine formulation described herein allows for the addition of an adjuvant with relative ease and without distorting the composition.

In addition, the vaccine of the present invention may be formulated so as to include a “depot” component to increase retention of the antigenic material at the administration site. By way of example, in addition to an adjuvant (if one is used), dextran sulfate or mineral oil may be added to provide this depot effect.

Immunogenicity of the vaccine formulation can be assessed by an in vitro opsonophagocytosis assay. For example, the immunogenicity of Type 5 and Type 8 specific antibodies generated by Type 5- and Type 8-rEPA conjugates can be evaluated as follows, using leukocytes (HL-60 promyelocytic leukemia cell line), complement, monoclonal or polyclonal CPS-specific antibodies and Type 5 or Type 8 bacteria. At time zero and 60 minutes opsonophagocytosis or killing of the bacteria is determined. A high correlation between the ELISA (enzyme-linked immunosorbent assay) detecting the presence of induced antibodies that bind Type 5 and Type 8 antigens, and the opsonic antibody activity for both Type 5 and Type 8 would indicate that the antibodies induced by the vaccine are functional and that they mediate type-specific opsonophagocytosis.

Additionally or alternatively, immunogenicity can be assessed by an animal assay such as described below in the examples. In such an assay, the level of antibodies induced in animals immunized with the vaccine is compared with the level of antibodies in non-vaccinated animals.

Exemplary formulations of the inventive vaccines comprising glycoconjugate immunogens comprise one or more Staphylococcal CPS antigens (such as S. aureus Type 5, S. aureus Type 8, S. aureus 336, and S. epidermis PS-1) conjugated to a carrier protein such as exotoxin A from Pseudomonas, tetanus toxoid or diphtheria. The CPS antigens typically comprise at least about 5% PG, determined as described above, but may comprise any amount of PG effective to enhance thiolation efficiency and/or immunogenicity without being unacceptably toxic For example, so long as the amount of PG does not reach a toxic limit that is unacceptable to the clinician, PG percentages higher than 10%, 15%, or 20% may be used.

Methods for Treating and Preventing Bacterial Infection

Also provided by the invention are methods for treating and/or preventing bacterial infection using a vaccine of the invention. Such methods comprise administering to a patient in need thereof a vaccine that comprises a therapeutically effective amount of a glycoconjugate immunogen, wherein (i) the glycoconjugate immunogen comprises at least one capsular polysaccharide and a carrier protein and (ii) the capsular polysaccharide comprises at least a minimum effective amount of PG, as described above. Typically, the vaccine also comprises a pharmaceutically acceptable carrier for the immunogen, as described above.

Target patient populations for these methods include mammals, including humans, who are infected with, or at risk of being infected by, bacterial pathogens, such a S. aureus or S. epidermis. The vaccine may be provided in any desired dosage form, including dosage forms that may be administered to a human intravenously, intramuscularly, or subcutaneously. The vaccine may be administered in a single dose, or in accordance with a multi-dosing protocol.

Administration may be by any number of routes, including subcutaneous, intracutaneous, and intravenous. In one embodiment, intramuscular administration is used. The skilled artisan will recognize that the route of administration will vary depending on the bacterial infection to be treated and the composition of the vaccine.

A vaccine according to the invention can be administered with or without an adjuvant. If an adjuvant is used, it is selected so as to avoid adjuvant-induced toxicity. A vaccine according to the invention may additionally comprise a β-glucan or granulocyte colony stimulating factor, in particular, a β-glucan as described in U.S. Pat. No. 6,355,625, filed Sep. 14, 1999 and issued Mar. 12, 2002.

A therapeutically effective amount of the vaccine of the present invention can be determined by methods that are routine in the art. Skilled artisans will recognize that the amount may vary with the composition of the vaccine, the particular patient's characteristics, the selected route of administration, and the nature of the bacterial infection being treated. General guidance can be found, for example, in the publications of the International Conference on Harmonisation and in REMINGTON'S PHARMACEUTICAL SCIENCES, chapters 27 and 28, at pages. 484-528 (Mack Publishing Company 1990). A typical vaccine dosage may range from 1 μg-400 μg.

The invention is further described by reference to the following examples, which are provided for illustration only. The invention is not limited to the examples but rather includes all variations that are evident from the teachings provided herein.

### EXAMPLES

### Example 1

### Isolation of a Type 5 Capsular Polysaccharide

Primary Enzymatic Digestion and Centrifugation

Type 5 CPS is released of from the bacterium as follows:

Type 5 CPS is purified from S. aureus by resuspending a Type 5 cell paste in Tris buffer. Lyostaphin is added at a final concentration of 16 units/gm of paste. At the end of 3 hours of lysostaphin digestion, RNAse and DNAse are added at a final concentration of 40 μg/mL of mixture to digest nucleic acids and reduce viscosity of mixture. This mixture is incubated for 3 hours at 37° C. with continuous stirring. The enzymatic mixture is centrifuged at 23,000 G for one hour and the supernatant is collected.

Primary Ethanol Precipitation, Centrifugation and Filtration

To remove the digested nucleic acids and other cellular components, dehydrated alcohol and CaCl₂ are added to the supernatant The solution is stored for 6-18 hours at 4° C. The 25% ethanol-precipitate is centrifuged and the pellet is discarded. This is followed by another precipitation to collect the crude CPS. Dehydrated ethanol and CaCl₂ are added and the solution stored for 6-18 hours at 4° C. The 75% ethanol precipitate is centrifuged, and the supernatant is discarded. The pellet is re-dissolved in water and filtered.

Dialysis and Filtration

The ethanol purified CPS is dialysed to remove traces of ethanol in dialysis tubing. The CPS is dialysed and the dialysate and retentate is tested for the presence of CPS by serotype identity testing using the capillary precipitation test.

In the capillary precipitation test, bacterial samples are lysed with a sucrose solution and incubated for fifteen minutes at room temperature and a portion of the lysed cells are diluted with water and incubated for fifteen more minutes. The sample is centrifuged, an aliquot of the supernatant is collected into a capillary tube, and an equivalent volume of specific antiserum is collected in a second tube. The contents of the antiserum tube are transferred into the sample tube and inspected under fluorescent light. The presence of a precipitate at the antiserum/sample interface is recorded as a positive result and the absence of a precipitate is recorded as a negative result. The tested retentate is filtered and lyophilized.

Secondary Enzymatic Digestion and Dialysis

To further purify the crude Type 5 CPS, the lyophilized material is dissolved in 0.05 M Tris with 2 mM MgSO₄, pH 7.2. Lyostaphin is added at a final concentration of 16 units/gm of paste. RNAse and DNAse is added at a final concentration of 100 μg/mL while in dialysis tubing (MW 10,000). This mixture is incubated for 4 hours at 37° C. The dialyzed mixture is tested for the presence of PS by serotype identity testing using the capillary precipitation test.

Secondary Ethanol Precipitation, Dialysis and Filtration

Dihydrated alcohol and CaCl₂ is added to the retentate from the dialysis tubing and the suspension is stored at 4° C. for 6-18 hours and centrifuged for one hour. The supernatant is collected and combined with dehydrated alcohol and CaCl₂. This suspension is centrifuged for one hour at 23,000 G. The pellet is dialyzed overnight to remove traces of ethanol. The dialysate and retentate are tested for the presence of CPS by serotype identity testing using the capillary precipitation test. The retentate is filtered through a 0.45 μm filter and stored for 6-18 hours at 4° C. The sample is lyophilized and stored until the next step.

Ion Exchange Chromatography

The sample is subjected to ion exchange chromatography for separation. The sample is applied to a DEAE column, and the column is washed five times with a flow rate of 60 mL/hr. Fractions of the effluent are monitored spectrophotometrically (OD₂₀₆).

Size Exclusion/Gel Filtration Chromatography

The lyophilized CPS is further purified by molecular size using size exclusion/gel filtration chromatography. The lyophilized material is dissolved in 0.2 M NaCl loaded onto the column and eluted with the same buffer. Fractions are collected and monitored at OD₂₀₆. Peak fractions are collected, tested for serotype identity as described above, filtered through a 0.45 μm filter and lyophilized.

The S4000 HPLC size exclusion chromatography (SEC) method is a qualitative procedure for the determination of molecular size of S. aureus capsular polysaccharide (CPS) using the Biosep-SEC-S4000 column. Each polysaccharide sample and marker is monitored at 206 nm and an area report is generated. The molecular size of a polysaccharide sample is represented as distribution coefficient (K_{d}), which is calculated from the column marker volumes and the sample elution volume, column void volume (2000 kD dextran), and total column volume (glycyl-L-tyrosine).

### Example 2

### Evaluation of CPS PG Content and Protein and Nucleic Acid Contamination

This example describes the quantitation of peptidoglycan amino acids and residual protein amino acids by amino acid analysis (AAA) and of nucleic acid contamination.

Procedure

Amino acid analysis (AAA) is used to determine the concentration of peptidoglycan and residual protein present in S. aureus polysaccharide samples. AAA of polysaccharide solutions is performed by hydrolyzing samples (prepared as 1 mg/mL purified polysaccharide in water) with vapor phase hydrochloric acid. Reconstituted primary and secondary amino acids are converted to stable fluorescent derivatives that fluoresce strongly at 395 nm. Analysis of the re-suspended protein hydrolysate is performed by reverse phase HPLC. The amino acids are quantified by means of external and internal standards. Amino acids are present in the polysaccharide solution arising from (1) peptidoglycan (residues Ala, Glx, Gly and Lys) and (2) residual proteins (residues Arg, Asx, Ile, Leu, Met, Phe, Ser, Thr, Thy, Val, His and Pro). Two amino acids (Cys and Trp) are not quantitated and are therefore not reported. The concentrations of the amino acids associated with peptidoglycan and residual protein are reported as a mass percentage relative to the polysaccharide using the following equations:

Calculation for % Peptidoglycan (w/w):
% Peptidoglycan=[PG]×100=[CPS]
[PG] mg/mL=Glu/Gln+Gly+Ala+Lys

where:
[protein]=protein concentration of sample (mg/mL) by amino acid analysis
[PG]=calculated peptidoglycan concentration of sample (mg/mL)
[CPS]=known polysaccharide concentration of sample (1 mg/mL)

For example, a vaccine formulation that comprises:
[PG] mg/mL=Glu/Gln+Gly+Ala+Lys
[PG] mg/mL=0.0119+0.0208+0.0132+0.0122=0.0581 mg/mL
[CPS]=0.96 mg/mL
contains 6.05% PG (% PG=[PG]/[CPS]×100=0.0581/0.96×100=6.05%).

Calculation for % Residual protein (w/w):
% RP=(peptides)_{cps}−(PG)/(cPS)×100
Σ(Amino acids)=(peptides)_{cps}

where:
(peptides)_{cps}=Total peptide concentration
[RP]=calculated residual protein concentration of sample (mg/mL)
[PS]=known polysaccharide concentration of sample (1 mg/mL)

For example, a vaccine formulation that comprises:
(peptides)_{cps}=0.0647 mg/mL
[PG]=0.0581 mg/mL
[cPS]=0.96 mg/mL
   contains 0.68% RP (% [RP]=0.0647-0.0581/0.96=0.68%)

Residual Nucleic Acid Quantitation by UV Spectrophotometry

The residual nucleic acid concentration of purified CPS can be determined by spectrophotometric analysis. The absorbance of a sample at 260 nm is compared to that of a solution of herring sperm DNA, which has an absorbance of 1.0 AU at 50 μg/mL. The concentration of DNA in the sample is reported as the fraction (%) of the total Type 5 polysaccharide concentration.

### Example 3

### Type 5 and Type 8 CPS Vaccine Efficiency

Type 5 and Type 8 CPS prepared as described in Example 1 above were determined to have the following properties:

| | Type 5 CPS | | Type 8 CPS | |
|---|---|---|---|---|
| TESTS | Batch 1 | Batch 2 | Batch 1 | Batch 2 |
| Molecular size (Kd) | 0.25 | 0.26 | 0.43 | 0.43 |
| Residual protein by amino acid | 0.028 | 0.21 | 0.68 | 0.45 |
| analysis (%) | | | | |
| Residual nucleic acid (%) | <1 | <1 | <1 | <1 |
| Peptidoglycan content (%) | 13.57 | 14.17 | 6.05 | 5.76 |
| Quantitation of CPS by ELISA | 94 | 103 | 80 | 102 |
| (μg/mL) | | | | |
| Potency in mice by ELISA | 66.5 | 93.6 | 82.9 | 77.8 |
| (μg/mL) | | | | |
| Potency in mice by responders (10 | 10/10* | 10/10 | 10/10 | 10/10 |
| mice/batch) | | | | |

*70% of mice exhibit ≧4X increase in antibody titer over control group.

As shown in the table, the Type 5 and Type 8 CPS were purified, determined to comprise at least 5% PG, and determined to be immunogenic in mice. The low level of residual protein lends confidence to the calculated peptidoglycan content because it shows that substantially all of the detected amino acids are contributed by peptidoglycan, not other residual protein. Assessment of the potency of the Type 5 and Type 8 CPS in mice is described in more detail below.

The potency of an S. aureus Type 5 and Type 8 CPS vaccine may be measured by mouse immunogenicity. The potency of the vaccine is determined by measuring the antibody response in individual mouse sera and by identifying the proportion of mice that demonstrates a significant (for example, four-fold) increase in antibody response. For example, the following procedure may be used:

Female, 6- to 8-week old mice are dosed in two groups of ten. Each group is vaccinated 2 times two weeks apart. The first group receives 0.25 μg vaccine per dose diluted to 100 μL with PBS/0.01% polysorbate 80. The second group of mice is a negative control group and receives 100 μL of PBS/0.01% polysorbate 80. Pre-vaccination blood samples are collected from selected mice in each group at least 48 hours prior to the first injection. Post-vaccination blood samples are collected from all mice one week after the last immunization. Sera samples are separated from whole blood samples by centrifugation.

Antibodies to S. aureus Type 5/8 polysaccharides are quantified in all sera samples by a quantitative ELISA. The samples are applied to microtiter plates coated with Type 5 or Type 8 CPS and incubated and washed with washing solution (0.01 M phosphate, 0.15 M sodium chloride, 0.1% v/v polysorbate 20) to remove any unbound murine antibodies. The amount of antibody remaining bound to the CPS is determined by subsequent reaction with goat anti-murine immunoglobulin (IgG) antibody conjugated to horseradish peroxidase (HRP). The level of bound HRP is determined by endpoint chromogenic reaction with 3,3′,5,5′-tetramethylbenzidine (TMB). The peroxidase activity is quenched by Stop Solution (1.0M phosphoric acid) and quantified by absorbance at 450 nm.

One measure of potency obtainable from this assay is the proportion of mice in the 0.25 μg dose group (%) that show a four-fold increase in both Type 5 and Type 8 serum antibody level relative to the geometric mean antibody level of the mice in the control group. For each mouse in the S. aureus vaccine groups, the fold increase in titer over the control group is the ratio of its serum antibody level to the geometric mean antibody level of the mice in the control group.

Another measure of potency obtainable from this assay is the geometric mean of the Type 5 and Type 8 antibody level (μg/mL) observed in the 0.025 μg dose group.

### Example 4

### Toxicology Studies

Single dose acute toxicity studies are conducted on a vaccine according to the present invention that comprises Type 5 and Type 8 capsular polysaccharides, where each CPS comprises at least 5% PG and is conjugated to rEPA, as follows:

TABLE 1
Toxicology studies conducted for the vaccine and vaccine components

| Study type and | | Route of | |
|---|---|---|---|
| duration | Study Number | administration | Species |
| Single dose toxicity | 1 | Intramuscular | Rat |
| Single dose toxicity | 2 | Intraperitoneal | Mouse |

Study 1: Single Dose Acute Toxicity Study (IM)

Three groups of 10 rats are administered a single dose of vaccine at low (2.92 μg/kg) or high (29 μg/kg) doses of the formulation in buffer. Half the animals in each group are sacrificed after 24 hours and the remainder are sacrificed 7 days following administration of the test and control articles. Test animals are evaluated for mortality, clinical signs, body weight, clinical pathology (hematology and clinical chemistry), gross pathology and histopathology. No treatment-related effects are observed for clinical signs, body weights, clinical pathology (hematology and clinical chemistry), gross pathology or histopathology. The study demonstrates that the vaccine does not induce toxic symptoms at the maximum tested dose, 29 μg/kg (20 times the human dose of 100 μg on a weight:weight basis).

Study 2: Single Dose Acute Toxicity Study (IP)

Eleven groups of 10 ICR mice are administered a single dose of the vaccine (25 μg type 5 and type 8 CPS), monovalent Type 5- and Type 8-rEPA conjugates (25 μg), rEPA (50 μg), Pseudomonas aeruginosa exotoxin A (0.1-0.5 μg) or bovine serum albumin. The test animals are observed for 48 hours after test article administration. Serum samples are measured for transaminase liver enzymes, SGOT and SGPT. Only exotoxin A causes mortality and elevated SGOT and SGPT levels. No abnormal clinical observations, unscheduled deaths or toxic effects are noted in the groups administered S. aureus CPS-rEPA vaccines or rEPA (approximately 900 times the human dose of 100 μg on a weight:weight basis for each material).

### Example 5

### Vaccine Formulation

A glycoconjugate vaccine according to the invention and comprising S. aureus Type 5 and Type 8 CPS was prepared. Typically, the vaccine formulation contains:

| | | |
|---|---|---|
| S. aureus Type 5 conjugate | 100 | μg |
| S. aureus Type 8 conjugate | 100 | μg |
| Polysorbate 80 | 0.1 | mg |
| Sodium chloride | 11.7 | mg |
| Sodium phosphate, dibasic | 223 | mg |
| Sodium phosphate, monobasic | 0.23 | mg |
| Water for injection | 1.0 | mL |

Testing of the purified CPS demonstrates the following:

| Molecular size | 0.16-0.34 Kd |
|---|---|
| Residual protein | <1% |
| Nucleic acid content by OD | <1% |
| O-acetyl content | >55% |
| Peptidoglycan content | at least about 5% |
| Thiol:CPS molar ratio | 0.05-.11 |

What is claimed is:

1. A vaccine, comprising:
(A) a therapeutically effective amount of a glycoconjugate immunogen comprising at least one capsular polysaccharide and a carrier protein, wherein said capsular polysaccharide comprises at least about 5% (w/w) peptidoglycan, based on the weight of the capsular polysaccharide, and
(B) a pharmaceutically acceptable carrier for said immunogen.

2. The vaccine of claim 1, wherein the glycoconjugate immunogen comprises a capsular polysaccharide expressed by Staphylococcus.

3. The vaccine of claim 2, wherein the glycoconjugate immunogen comprises one or more capsular polysaccharides selected from the group consisting of capsular polysaccharides expressed by Staphylococcus aureus and capsular polysaccharides expressed by Staphylococcus epidermis, wherein at least one capsular polysaccharide comprises at least about 5% (w/w) peptidoglycan.

4. The vaccine of claim 2, wherein the capsular polysaccharide is selected from the group consisting of a Type 5 capsular polysaccharide, a Type 8 capsular polysaccharide, 336 capsular polysaccharide, PS-1 capsular polysaccharide, and combinations thereof, wherein at least one capsular polysaccharide comprises at least about 5% (w/w) peptidoglycan.

5. The vaccine of claim 1, wherein the carrier protein is selected from the group consisting of exotoxin A from Pseudomonas, tetanus toxoid, diphtheria, alpha hemolysin, and Panton-Valentine leukocidin.

6. The vaccine of claim 4, wherein said capsular polysaccharide comprises a Type 5 capsular polysaccharide and a Type 8 capsular polysaccharide.

7. The vaccine of claim 4, comprising (i) a Type 5 capsular polysaccharide conjugated to an exotoxin A carrier protein from Pseudomonas and (ii) a Type 8 capsular polysaccharide conjugated to an exotoxin A carrier protein from Pseudomonas.

8. The vaccine of claim 4, comprising a 336 capsular polysaccharide.

9. The vaccine of claim 4, comprising a PS-1 capsular polysaccharide.

10. The vaccine of claim 4, wherein said capsular polysaccharide comprises a 336 capsular polysaccharide and a PS-1 capsular polysaccharide.

11. A method for treating a bacterial infection, comprising administering a vaccine of claim 1.

12. A method for making a vaccine comprising a glycoconjugate immunogen comprised of at least one capsular polysaccharide and a carrier protein, comprising:
(A) conjugating at least one capsular polysaccharide to a carrier protein to form a glycoconjugate immunogen, wherein the capsular polysaccharide comprises at least about 5% (w/w) peptidoglycan, based on the weight of the capsular polysaccharide and
(B) formulating a therapeutically effective amount of the glycoconjugate immunogen with a pharmaceutically acceptable carrier for said immunogen.

13. A method for enhancing the conjugation efficiency of a capsular polysaccharide to a carrier protein, comprising (i) selecting capsular polysaccharide that comprises an amount of peptidoglycan effective to contribute to enhanced conjugation efficiency of the capsular polysaccharide and carrier protein, and (ii) conjugating the capsular polysaccharide to a carrier protein.

14. The method of claim 13, wherein the capsular polysaccharide comprises an amount of peptidoglycan effective to increase the conjugation efficiency of the capsular polysachharide to the carrier protein by at least about 20% relative to capsular polysaccharide comprising about 2% peptidoglycan.

15. The method of claim 13, wherein the capsular polysaccharide comprises at least about 5% (w/w) peptidoglycan, based on the weight of the capsular polysaccharide.

16. A method for enhancing the immunogenicity of a vaccine, comprising (i) selecting capsular polysaccharide comprises an amount of peptidoglycan that contributes to enhanced immunogenicity of the vaccine, (ii) conjugating the capsular polysaccharide with a carrier protein to form a glycoconjugate immunogen and (iii) preparing a vaccine comprising the glycoconjugate immunogen and a pharmaceutically acceptable carrier.

17. The method of claim 16, wherein the capsular polysaccharide comprises at least about 5% (w/w) peptidoglycan, based on the weight of the capsular polysaccharide.

18. A vaccine, comprising:
(A) a therapeutically effective amount of a glycoconjugate immunogen comprising at least one capsular polysaccharide and a carrier protein, wherein said capsular polysaccharide comprises an amount of peptidoglycan effective to increase the conjugation efficiency of the capsular polysaccharide to the carrier protein by at least about 20% relative to capsular polysaccharide comprising about 2% peptidoglycan, and
(B) a pharmaceutically acceptable carrier for said immunogen.
